(19) United States
(12) Patent Application Publication
(10) Pub. No.: US 2012/0157819 A1
(43) Pub. Date: Jun. 21, 2012

(54) IMAGING METHOD AND IMAGING DEVICE FOR DISPLAYING DECOMPRESSED VIEWS OF A TISSUE REGION

(75) Inventors: ANNA JEREBKO, ERLANGEN (DE); DANIEL FISCHER, ERLANGEN (DE)

(73) Assignee: SIEMENS AKTIENGESELLSCHAFT, MUENCHEN (DE)

(21) Appl. No.: 13/333,076

(22) Filed: Dec. 21, 2011

(30) Foreign Application Priority Data
Dec. 21, 2010 (DE) 10 2010 063 810.2

(51) Int. Cl. A61B 6/00

(52) U.S. Cl. 600/407

(57) ABSTRACT

A method visualizes a tissue region. The method includes the following steps: inserting the tissue region into the capturing region of a first imaging modality, with the tissue region assuming a first shape; capturing the interior of the tissue region by the first imaging modality; establishing a first image volume of the interior of the tissue region when it assumes the first shape; and first transforming of the first image volume into a second image volume, which represents a surface and interior regions of the tissue when the tissue region assumes a second shape.

### CROSS-REFERENCE TO RELATED APPLICATION

This application claims the priority, under 35 U.S.C. §119, of German application DE 10 2010 063 810.2, filed Dec. 21, 2010; the prior application is herewith incorporated by reference in its entirety.

### BACKGROUND OF THE INVENTION

### Field of the Invention

The present invention relates to an imaging method and an imaging device for displaying decompressed views of a tissue region, more particularly the mamma, which take into account the compression of the mamma during the image recording and generate decompressed views of the mamma.

In a tomosynthesis method, a three-dimensional image is generated from a plurality of two-dimensional images. An X-ray apparatus with an X-ray beam source and a detector is used to generate a first two-dimensional image or a first projection of the tissue to be examined, through which tissue the X-ray beam passes. Here, the two-dimensional image represents the attenuation of the X-ray radiation by the tissue in the volume or in the breast. A second two-dimensional image or a second projection of the same tissue or volume is recorded after the beam source and/or the detector was moved into a second position. After a plurality of two-dimensional images was recorded, a three-dimensional tomosynthesis image can be generated by a reconstruction.

Mammography is a field of application of the three-dimensional imaging method mentioned at the outset. An image generation device typically used in mammography contains a pivotable X-ray beam source and a stationary X-ray detector. The tissue to be examined is positioned over the stationary detector, with the tissue to be examined being compressed and not being in its natural shape. The X-ray source is subsequently pivoted over a number of steps or continuously, for example within a range of +/−25°, and the stationary detector is used to record a plurality of two-dimensional X-ray images from different pivot positions of the X-ray beam source. It goes without saying that it is also possible to use a plurality of stationary X-ray beam sources or to merely displace the X-ray beam source in a translational fashion. It is also possible for the detector to be displaced or pivoted counter to the movement of the X-ray source. In the case of craniocaudal recordings, the X-ray beam source(s) emit(s) X-ray beams from positions that are arranged along a line running parallel to the axis running from one shoulder of the patient to the other. A beam path parallel to the chest wall can result in the entire tissue of the breast being imaged and the thorax not being irradiated. A three dimensional image is generated from the plurality of two-dimensional X-ray images by the reconstruction. Imaging methods and devices for mammography from the prior art are for example described in published, non-prosecuted German patent applications DE 10 2006 046 741 A1 (corresponding to U.S. patent publication No. 20100020920), DE 10 2008 004 473 A1 (corresponding to U.S. patent publication No. 20100034450), DE 10 2008 033 150 A1 (corresponding to U.S. patent publication No. 20110122992), DE 10 2008 028 387 A1 (corresponding to U.S. patent publication No. 20090310844) and published European patent application EP 2 138 098 A1 (corresponding to U.S. Pat. No. 7,965,812).

In the prior art, so-called filtered back projections are used to reconstruct a three-dimensional image from a plurality of two-dimensional images; by way of example, these filtered back projections are described in chapter 10.5 of Imaging Systems for Medical Diagnostics, Arnulf Oppelt, Publicis Corporate Publishing, Erlangen, ISBN 3-89578-226-2. These filtered back projection reconstruction methods display reconstructed images with a comparatively high contrast and comparatively great detail, but lose information in respect of the relative tissue density in the case of tomosynthesis with a restricted scanning angle as a result of the missing data. This is the result of certain filter kernels removing low-frequency components. In general, digital breast tomosynthesis (DBT) is afflicted by incomplete data and poor quantum statistics, which is restricted by the overall dose absorbed in the breast. The breast mainly consists of glandular tissue, fatty tissue, connective tissue and blood vessels. The X-ray attenuation coefficients of these types of tissue are very similar, making the evaluation of three-dimensional mammography images significantly harder. The main field of application of imaging methods in mammography is the early detection of cancerous tissue. This is made more difficult by the fact that cancerous tissue has a similar X-ray attenuation coefficient to other types of tissue.

By way of example, mammography methods are described in chapter 12.6 of Imaging Systems for Medical Diagnostics, Arnulf Oppelt, Publicis Corporate Publishing, Erlangen, ISBN 3-89578-226-2. The breast is usually scanned in two positions, namely craniocaudal and mediolateral oblique, while it is compressed on a table 6 by a compression plate 4 (see FIG. 1). The average thickness of the compressed breast 2 is approximately 4 cm. Hence the spatial association between the breast itself and the location of the tissue changes (lesions) is distorted compared to the non-compressed breast. Compressing the breast 2 improves the visibility of tissue changes, but makes it more difficult to interpret the location of the tissue change and, in particular, to estimate the location of the lesion in the non-compressed breast is made more difficult, which location is required for planning the operation.

If breast cancer is discovered in the breast of a patient and a surgical intervention is planned, the radiologist must inform the surgeon about the position of the tissue change(s). The radiologist usually marks the tissue change(s) in the two-dimensional image data and/or draws an approximate schematic illustration. Moreover, some radiologists use three-dimensional imaging methods, e.g. CT, MRI or tomosynthesis. The surgeon plans the surgical intervention on the basis of this information in respect of the location of the tissue change or the locations of the tissue changes.

Imaging methods such as mammography or conventional ultrasound methods merely generate two-dimensional images. Additionally, the breast is deformed during the image recording. By way of example, in the case of mammography by means of tomosynthesis, the breast is compressed by the compression plate. In the case of magnetic resonance imaging, the breast is deformed by gravity and the body weight if the patient lies face down. During the intervention, the patient usually lies on her back, and only gravity acts on the breast. Therefore the surgeon often finds it difficult to determine the location of the tissue changes during the planning procedure and the operation from the image data of the compressed and deformed breast.

Currently surgeons and radiologists estimate the location of the tissue change on the basis of a schematic two-dimensional image and the distance from the nipple or mamilla.

### SUMMARY OF THE INVENTION

It is accordingly an object of the invention to provide an imaging method and an imaging device for displaying decompressed views of a tissue region which overcomes the above-mentioned disadvantages of the prior art devices and methods of this general type, such that estimating the location of tissue changes can be improved.

The method for visualizing the interior of a tissue region includes the insertion of the tissue region into the capturing region of a first imaging modality, with the tissue region assuming a first shape. The interior of the tissue region is captured by a first imaging modality. A first image volume of the interior of the tissue region is established when it assumes the first shape. A first transformation of the first image volume into a second image volume is illustrated, which transformation represents the interior of the tissue when the tissue region assumes a second shape. The second shape is simulated by the method.

The tissue region can be the mamma. The first imaging modality can be a digital breast tomosynthesis (DBT) system, in which the breast of a patient is compressed between a compression plate and a compression table. The interior of the tissue region can be captured by X-ray recordings from a plurality of projection directions. The volume can be displayed in three dimensions using slice images. The second shape of the tissue region can be the shape in which the patient lies on the back or the patient lies on the stomach and the breast hangs through an opening in the couch, with no further pressure being exerted on the tissue or the breast.

It is possible to determine the volume assumed by the tissue in its first shape. It is possible to determine the tissue density or tissue densities of the tissue region from the first image volume when the tissue assumes its first shape. As an alternative to this, or in addition thereto, it is possible to determine the force acting on the tissue when it assumes the first shape. During the first transformation, it is possible to take account of the tissue volume, the tissue density (tissue densities) and/or the force acting on the tissue region.

The force acting on the tissue and the volume of the tissue region can be determined when the tissue region is in the first imaging modality. These parameters serve as input parameters during the first transformation. The tissue density (tissue densities) can be determined from the first image volume, as determined by the first imaging modality. The tissue density can also be an input parameter for the first transformation.

A first partial image volume can be marked in the first image volume. The first partial image volume can be displayed at a corresponding position in the second image volume. The marked first partial image volume can be changed tissue, for example cancerous tissue. The step of marking can be brought about by a user or an automatic method, for example an automatic segmentation by thresholding. If the position of the first partial image volume, i.e. the position of the changed or damaged tissue, is displayed in a corresponding position in the second image volume, the surgeon can plan the operation more reliably because the position of the damaged tissue can be determined more accurately.

The second image volume can be transformed into a third image volume, in which the tissue region assumes a third shape. A direct transformation from the first image volume to the third image volume is also possible. The tissue region can assume the third shape when the patient is in the supine position and gravity shapes or deforms the tissue region differently than is the case if the tissue region assumes the first or second shape.

The step of the first transformation can simulate a decompression of the first image volume and/or the step of the second transformation can simulate a compression of the second image volume. During the first transformation, the tissue region is transformed from a first shape, into which it is compressed by the first imaging modality, into a second shape, in which the patient lies on the back or the stomach and the breast hangs through an opening. During the second transformation, the image volume is transformed from a second shape into a third shape, in which the patient is in a supine position, as required for an operation, for example.

The method can furthermore contain the step of generating a mesh representation from the second and/or the third image volume. The step of the first transformation and/or the step of the second transformation can comprise the generation of slice images, and so the second image volume and/or the third image volume has slice images.

In the step of the first transformation and/or the second transformation, the method can use a model, for example a model of the breast, which is selected from a plurality of models as a model with the best correspondence. The model can have first image volume model data which is associated with the first shape of the tissue region, second image volume model data which is associated with the second shape of the tissue region, and/or third image volume model data which is associated with the third shape of the tissue region. The model can be determined using a plurality of imaging modalities.

The method can extract characteristic features of the tissue region, for example when the tissue region is in the first shape. The model can be determined on the basis of the change in the position and/or the size of the characteristic features when the tissue region assumes the first shape, the second shape and/or the third shape. A set of non-compressed breast models can be calculated in advance using MRI data when the patient lies on her stomach and the breasts hang through a hole in the patient couch. Moreover, the breasts can be placed into a water bath during the MRI recording for generating a model so that no gravitational force acts on the breast (the breast is then not subjected to forces). Furthermore, it is possible to calculate in advance a set of non-compressed breast models using MRI data or CT data when the patient is in a supine position (for example for thorax examinations). The set of MRI data or CT data used for determining the model can comprise DBT images and/or full field digital mammography (FFDM) images for the same patients, which were determined at approximately the same time (+/−3 months). All data records should be obtained prior to a possibly necessary intervention. The models can be determined from manually or (semi-)automatically segmented breast recordings, which are obtained from an MRI volume image or a CT volume image by building up a regular or irregular breast surface mesh or from a three-dimensional volume with an constant intensity (in respect of the voxel value) within the breast and a different intensity outside of the breast. The resolution of the three-dimensional volume or a surface grid can be reduced such that they require minimal storage space. Use can be made of the lowest acceptable resolution.

The model can determine a breast which is decompressed virtually, for example the second shape of the tissue region, on the basis of features that are based on the breast size, breast shape, compressed thickness and/or the composition thereof, which are automatically determined from one or more DBT view(s) and/or from at least one FFDM view or which are determined during the recording.

In other words, a plurality of first image volumes from different patients is observed when the tissue region is in the first shape. It is possible to extract characteristic features in the first image volume. The second image volume of the same plurality of patients is observed thereafter when the tissue region assumes the second shape. The model is generated from the change between the first image volume and the second image volume from one patient of the plurality of patients. To this end, use can be made of the change in the characteristic features, as well as the size, location and/or orientation thereof. This model can be used during the first transformation. The same method or the same procedure can be used to determine a further model, which is used for the step of the second transformation.

It is possible to calculate a set of N features which allows determination of the breast elasticity and/or estimation of the deformations. The features comprise the breast density (densities), the breast composition, the thickness of the compressed breast, the shape of the breast and/or the age of the patient; however, they are not restricted to these. These features are calculated from one or more DBT view(s) and/or FFDM view(s) or can be captured and buffered while the images are recorded. This set of features is matched for all DBT data records and FFDM data records to corresponding MRI data records and/or CT data records, which were used to calculate the set of decompressed breast models as per the second shape. The same sets of features are then determined for any new data record for which a decompressed breast model, the second image volume or the third image volume should be determined. These features can be used both when the model is formed and when image data from a patient is associated with a suitable model.

The most suitable breast model is used during the step of the first transforming and/or the second transforming. The suitable model can be selected on the basis of at least some of the characteristic features mentioned here. By way of example, the most suitable decompressed breast model from the model set is used for any new DBT data record or FFDM data record of a model, which is based on the closeness or similarity of criteria determined by a linear combination or a nonlinear combination of the aforementioned features. As an alternative to this, use can be made of the Euclidean distance in a one-dimensional feature space or a weighted distance, with the weightings being determined by a training algorithm or by machine learning. As an alternative to this, use can be made of various algorithms that are based on a nearest neighbor search.

In other words, the model can be determined by image volume data, which is determined by MRI and/or CT and matched to sets of features that were determined from DBT image volumes and/or FFDM images for the same patients. Each patient in the training set or model set should be associated with a first image volume when the breast is in the first shape, for example a compressed shape, determined by DBT images and/or FFDM images, and MR image data and/or CT image data, i.e. a second image volume, when the breast is in the second shape, for example in the decompressed shape.

When a patient is examined, all that is required now is a first image volume when the breast is in the first shape, which was for example established by DBT. Subsequently, the most suitable model can be used for the transformation into the second image volume in which the breast is in the second, e.g. decompressed, shape. This model can be used during the first transforming and/or second transforming. Put simply, image volume model data of a patient with the greatest correspondence in the DBT recordings or FFDM recordings in respect of the thickness of the compressed breast, the breast density, the size, etc. are sought after in the model set and then the surface grid model or the mesh drawing thereof, which was determined by CT or MRI, is used to calculate the second and/or third image volume of the new patient when the breast is in its second or third shape.

The model can be selected on the basis of characteristic features. The characteristic features can comprise the thickness of the compressed breast, the density of the breast and/or the size of the breast.

The invention also relates to an imaging system with a transformation apparatus configured to carry out the steps of the above-described method.

The invention also relates to surgical surroundings with at least one modality and the aforementioned imaging device.

Furthermore, the invention discloses a computer program product, which can be loaded into a memory of a computer or is stored therein, with measures configured to execute the steps of the aforementioned method.

Other features which are considered as characteristic for the invention are set forth in the appended claims.

Although the invention is illustrated and described herein as embodied in an imaging method and an imaging device for displaying decompressed views of a tissue region, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, perspective view of a modality in which a breast is compressed in order to carry out recordings of an interior of the breast according to the invention;

FIG. 2 is an illustration schematically showing a tomosynthesis being carried out;

FIG. 3 is an illustration showing a shape of the breast resulting from the compression in the modality;

FIG. 4 is an illustration showing slice images produced by use of DBT;

FIG. 5 is an illustration of an MRI recording of the breast;

FIG. 6 is a schematic illustration of the breast with a changed tissue when a patient is in a supine position;

FIG. 7 is a flowchart illustrating an imaging method according to the invention;

FIG. 8 is an illustration of a clock-face-like representation of the breasts;

FIG. 9 is a flowchart explaining a generation of a model; and

FIG. 10 is a schematic illustration of a device according to the invention.

### DETAILED DESCRIPTION OF THE INVENTION

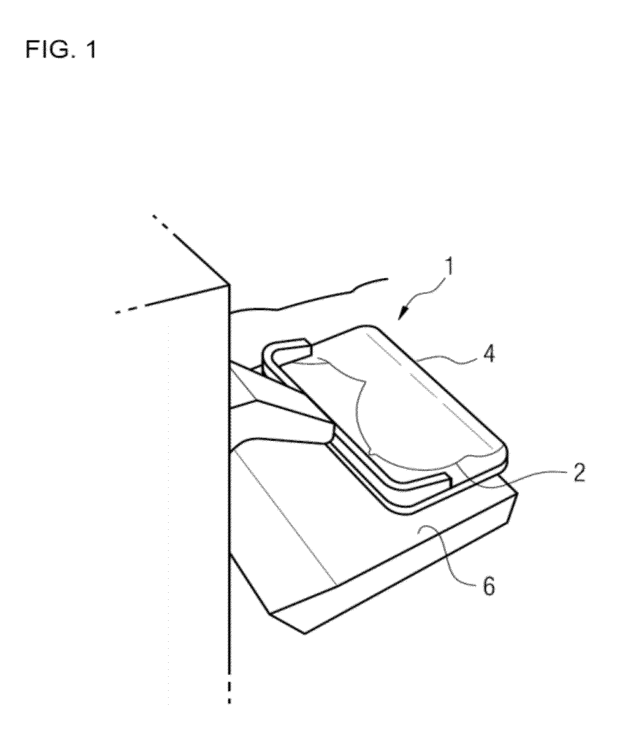

Referring now to the figures of the drawing in detail and first, particularly, to FIG. 1 thereof, there is shown a first imaging modality, which has a compression plate 4 and a compression table 6, between which a breast 2 is clamped. The breast is usually compressed in this fashion until a predetermined compression force is reached. A plurality of X-ray sources or at least one moveable non-illustrated X-ray source can be arranged over the compression plate 4. An X-ray detector can be arranged in or below the table 6. The device can be used to capture projections from different directions by X-ray radiation, from which, as described at the outset, it is possible to generate slice images. The functionality of a DBT-type modality was described at the outset and is known to a person skilled in the art from, for example, Imaging Systems for Medical Diagnostics, Arnulf Oppelt, Publicis Corporate Publishing, Erlangen, ISBN 3-89578-226-2; hence it is not described in any more detail.

The compressed breast is captured in the mediolateral oblique (MLO) position in the first imaging modality 1.

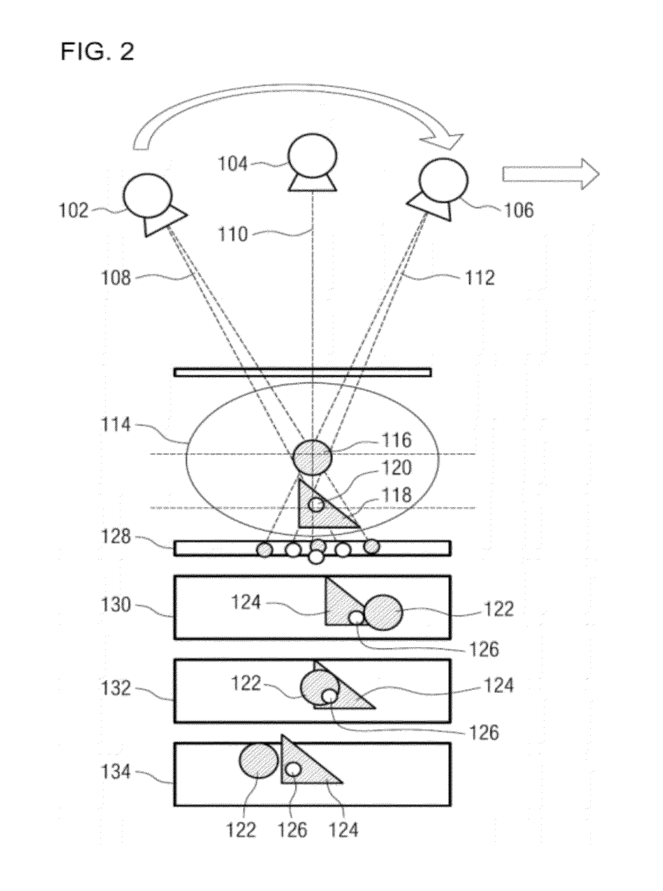

The method for generating the projections is explained with reference to FIG. 2. A plurality of X-ray sources 102, 104, 106 is arranged over an angular range of approximately 50°. It is possible for 25 X-ray sources to be arranged, and so 25 projections can be generated. As an alternative to this, it is also possible for an X-ray source to be pivoted over an angular range of 50° such that 25 projection recordings are generated. The first X-ray source 102 emits a first X-ray beam 108, which passes through the breast 114 and is attenuated by a first tissue region 116, a second tissue region 118 and a third tissue region 120. A detection element generates a first projection recording 130, in which a first tissue region image 122, a second tissue region image 124 and a third tissue region image 126 are in a first arrangement. The second X-ray beam source 104 emits a second X-ray beam 110, at a different angle, to the breast 114, the first tissue region 116, the second tissue region 118 and the third tissue region 120. These tissue regions are recorded by the second projection recording 132 and are in an arrangement that differs from that of the first projection recording 130. The third X-ray source 106 emits a third X-ray beam 112 to the breast at a further angle and the beam generates a third arrangement of the first tissue region image 122, the second tissue region image 124 and the third tissue region image 126 in the third projection recording 134.

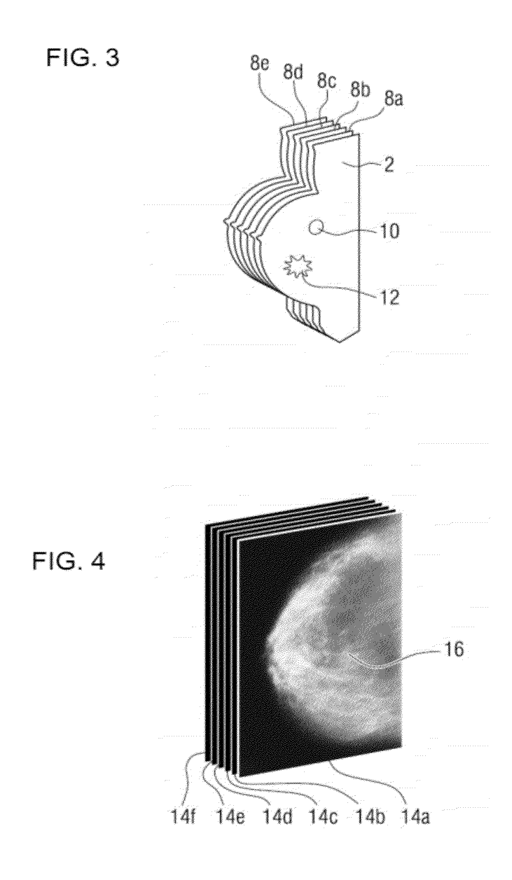

FIG. 3 shows the shape of the captured compressed breast, resulting from the compression, in the mediolateral oblique position. A plurality of slices 8a to 8e is shown. Furthermore, a first tissue change 10 and a second tissue change 12 are shown. Tissue changes can be identified comparatively well in the case of a compressed breast.

FIG. 4 shows a plurality of slice images captured by the first imaging modality, i.e. by use of DBT, which slice images form the first image volume of the breast 16. A plurality of slice images 14a to 14f is shown. The location of a tissue change (shown in FIG. 3) can be marked manually or automatically in these recordings. Since the breast is compressed in the mediolateral oblique position, the surgeon can only draw limited conclusions from the slice images 14a to 14f in respect of where the tissue changes 10, 12 are when the patient is in the supine position.

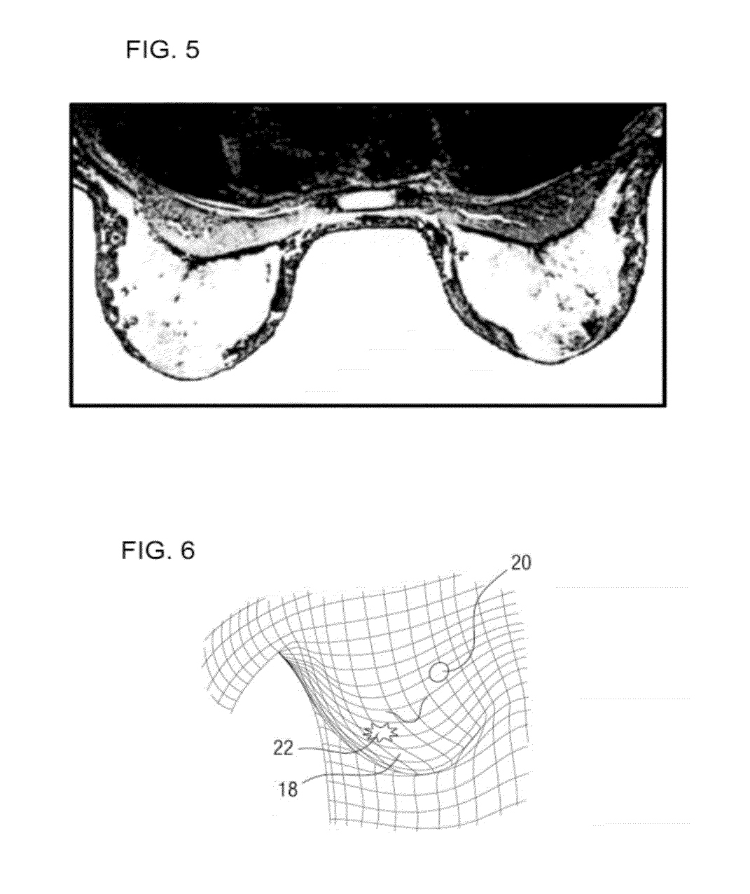

FIG. 5 shows a slice of an MRI volume, in which a woman lies on the stomach, with the breasts hanging through openings in the patient table.

FIG. 6 shows a mesh representation of the breast 18 of a patient who is lying on her back, corresponding to the conventional surgical position. The transformation according to the invention was used to transform the first image volume of the compressed breast 16 into a second image volume of the non-compressed breast 18. The tissue changes 20, 22 are shown to the surgeon in the mesh representation as per FIG. 4 such that the position thereof can be estimated prior to carrying out the intervention.

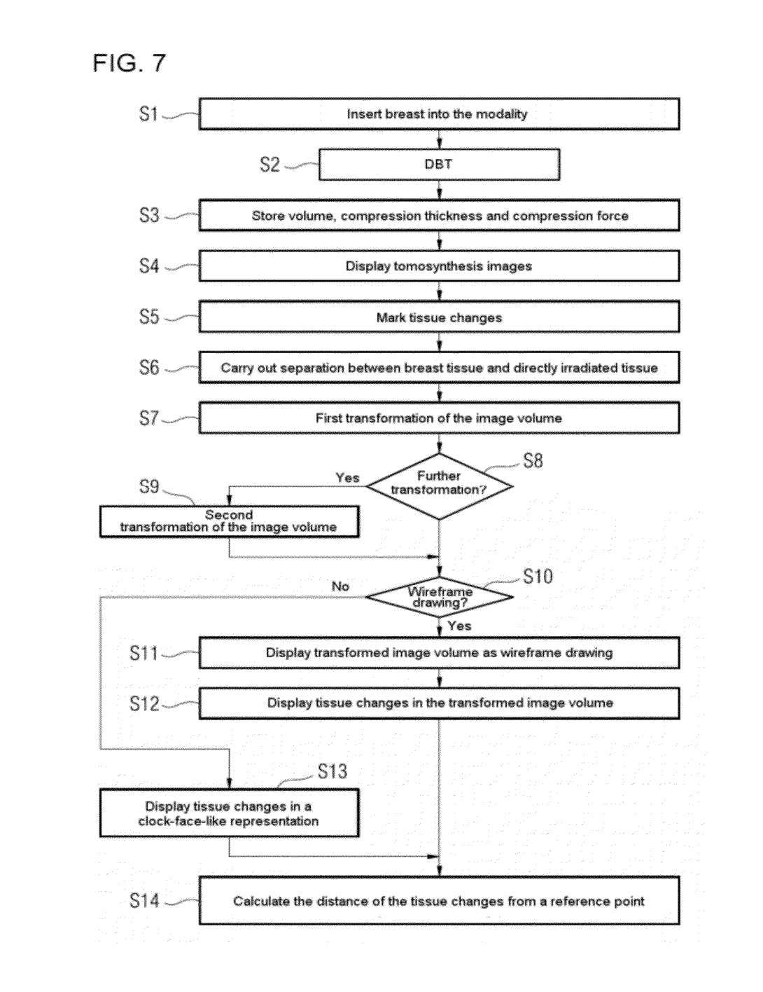

The method according to the invention will be explained in more detail below with reference to FIG. 7. In step S1 the breast is inserted into a first imaging modality, e.g. into a DBT system, where it is compressed to a predetermined thickness between a compression plate 2 and a compression table 6 (FIG. 1). The above-described DBT image recording method is carried out in step S2. To this end, a plurality of projections is generated by irradiating the breast with X-ray radiation from different angles. A first image volume is generated from projection images by a reconstruction method. Furthermore, at least the compression thickness and the compression force are stored in step S3.

In step S4, the tomosynthesis images are displayed on a monitor as slice images 14a to 14f of the breast 16 (FIG. 3). The number of slices can vary depending on breast thickness and reconstructed slice spacing. In step S5, tissue changes are marked in the tomosynthesis images. By way of example, the tissue changes contain cancerous tissue, carcinomas, lumps or other pathologically changed tissue. The tissue changes can be marked manually by a user, for example a radiologist. The tissue changes can be marked automatically by a computer-assisted identification mechanism. Furthermore, the region of the tissue change can be marked semi-automatically by virtue of a radiologist selecting a point with a tissue change and the complete tissue change can subsequently be marked by use of a segmentation algorithm.

In step S6, the breast tissue and the directly irradiated tissue are separated. Here use can be made of a segmentation algorithm which, for example, is based on thresholding. The literature has disclosed very different methods. An example is found in “Automated Segmentation of Digitalized Mammograms”, Ulrich Bick et al., Acta Radiol 1995.

A first transformation of the captured first image volume, i.e. the breast 16, which is illustrated in a plurality of slice images 14a to 14f (see FIG. 3), is carried out in step S7. In step S7, it is possible to carry out a virtual decompression of the first image volume, which was determined by the first imaging modality. The step of the first transformation can transform the first image volume in a case where it is simulated that the patient lies on her back. As a result, the first image volume is transformed into a second image volume.

In step S8, the user is asked whether a further transformation is to be carried out. If a further transformation of the image volume which is decompressed virtually is to be carried out, the method continues with step S9. In step S9 a second transformation of the image volume is carried out. The image volume can be transformed into a further image volume, for example a third image volume, which simulates the case in which the patient lies on her back. This representation of the image volume makes it significantly easier for a surgeon to plan and carry out the operation. After step S8 or after step S9, the method continues with a step S10, in which the user can determine whether a mesh representation of the transformed image volume is to be displayed. If a mesh representation of the transformed image volume is to be displayed, the method continues in step S11, in which the requested mesh representation is generated from the transformed image volume. The mesh representation allows a comparatively good illustration of three-dimensional spatial conditions in a two-dimensional representation. In step S12, which follows step S11, the previously marked tissue changes are illustrated in the transformed image volume. This makes it significantly easier for a surgeon to plan and carry out the operation. Should the user select in step S10 that no mesh representation is to be generated, the breast tissue and the marked tissue changes are, in step S13, shown in a clock-face-like representation. After step S12 or step S13, the method can in step S14 calculate and/or display the distance of the tissue change from a reference point. By way of example, the reference point can be the nipple.

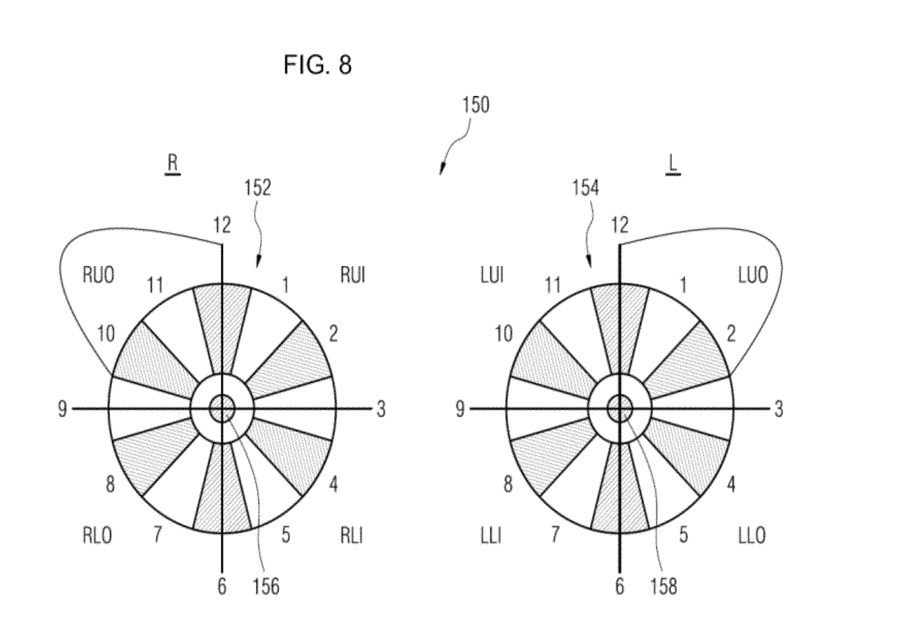

FIG. 8 schematically shows a clock-face-like representation 150 of the breasts. The first diagram 152 represents the right breast of a patient. The breast regions are subdivided into twelve partial regions 1 to 12, which are respectively divided amongst the quadrants RUI, RLI, RLO and RUO. The right nipple 156 is situated in the center of the first diagram. The second diagram 154 shows the left breast in a clock-face-like representation. The breast is subdivided into 12 segments 1 to 12. The 12 partial regions are divided amongst the 4 quadrants LUO, LLO, LLI and LUI.

Furthermore, it is possible to print out a report with the mesh representation and the tissue change(s) marked therein or a report in the form of print-outs of the clock-face-like representation of the breast tissue and the tissue change(s).

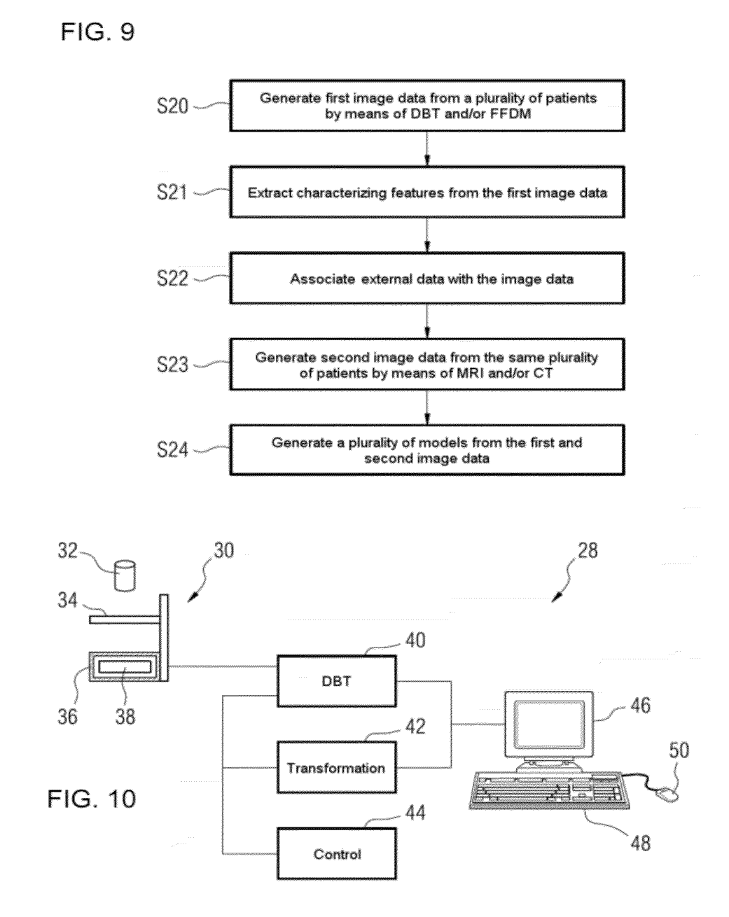

With reference to FIG. 9, the generation of at least one model is explained, which model can be used for the step of the first and/or second transformation. In step S20, first image data of a plurality of patients is generated by DBT and/or FFDM. In the optional step S21, characteristic features are extracted from the first image data and/or associated with the latter. By way of example, the characteristic features comprise the size of the breast, the shape of the breast, the thickness of the breast after compression etc. The characteristic features can be calculated automatically from the first image data or associated with the first image data as measurement values. A separate set of first image data can be generated for each patient. The characteristic features and/or external data (measurement values) are associated with the image data in step S22.

In step S23, the second image data of the same plurality of patients is generated by MRI and/or CT. The first and the second image data should be generated as closely together in time as possible, preferably within a timeframe of approximately 3 months. Both the first image data and the second image data should be generated prior to a possibly required intervention.

A plurality of models is generated from the first and second image data in step S24. To this end, the first image data of a patient is associated with the second image data of the same patient. The first image data can illustrate the breast in its first, compressed shape. The second image data can illustrate the breast in its second shape when the patient is in the supine position, lies on her stomach or is standing.

The models can be determined from manually or (semi-)automatically segmented breast recordings that are obtained from an MRI volume image or a CT volume image by building up a regular or irregular breast surface grid or from a three-dimensional volume with an unchanging intensity (in respect of the voxel value) within the breast and a different intensity outside of the breast. A segmentation mask can be used to this end. The resolution of the three-dimensional volume or a surface grid of the model can be reduced such that these require minimal storage space. Use can be made of the lowest acceptable resolution. The model can determine a breast which is decompressed virtually, for example the second shape of the tissue region, on the basis of features based on the breast size, breast shape, the compressed thickness and/or the composition thereof, which are automatically determined from one or more DBT view(s) and/or at least one FFDM view.

The age of the patient and the thickness of the breast during the compression can be obtained from the meta data of the digital imaging and communications in medicine (DICOM) image or the accompanying patient data, for example from a radiological information system (RIS) system or a hospital information system (HIS) system. This set of features is used for all DBT and FFDM data records with the corresponding MRI or CT data of the same patient, which were used to calculate the set of second image data, i.e. the non-compressed image data. The models can be stored in a database. The models are preferably calculated and stored for a plurality of different embodiments and sizes of the breast.

The following text describes the use of a model in the case of a patient for whom merely DBT image data and/or FFDM image data is available. By way of example, the model can be used during the step of the first transforming as per step S7 in FIG. 7 and/or during the step of the second transforming as per step S9 in FIG. 7. The most suitable model is selected from the aforementioned features and external data on the basis of the image data generated by DBT and/or FFDM. To this end, use can be made of similarity criteria or distance criteria, for example the Euclidean distance or a weighted distance. Use can also be made of learning algorithms (machine learning). In conclusion, a breast model based on CT or MRI can be associated with an image volume of a new patient on the basis of the thickness of the compressed breast, the breast thickness, the size, etc., wherein the image volume of the new patient was generated by DBT and/or FFDM. Furthermore, use can be made of algorithms for determining the nearest neighbor.

It is possible to calculate a set of N features which allows determination of the breast elasticity and/or estimation of the deformation of the breast. The features contain the breast density, the breast composition, the thickness of the compressed breast, the shape of the breast and/or the age of the patient; however, they are not restricted to these. These features are calculated from one or more DBT view(s) and/or FFDM view(s) or are associated with these. This set of features is matched for all DBT data records and FFDM data records to corresponding MRI data records and/or CT data records, which were used to select a decompressed breast model as per the second shape.

The changed tissue regions, which are shown in one or more DBT views and/or FFDM views, are illustrated in the transformed image volume (step S12 in FIG. 7). The transformed image volume can be displayed as a mesh volume or by a rotated volume in order to provide the surgeon with sufficiently precise data for planning the intervention. Moreover, it is possible to use the distances from the tissue subregion (e.g. a lesion) to characteristic features, for example to the nipple, to the thoracic muscles, to the transition between breast and chest, the distance to the plane of the detector and/or the distance to the compression plate in order to estimate the location of the tissue change in the case of the non-compressed breast, i.e. the transformed image volume, when the breast assumes the simulated second shape. The estimated location of the tissue of a breast which is decompressed virtually (transformed image volume) can be refined if a plurality of DBT slice images and/or FFDM views are available, in which the tissue change is displayed. When the breast which is decompressed virtually (transformed image volume) is displayed, accuracy ranges can be displayed for the estimated locations of the tissue changes. Furthermore, the distance from the tissue change(s) to the nipple can be displayed.

The transformation and the display of the changed tissue region after the transformation in the breast which is decompressed virtually can take place more or less in real time, with processing times of a few seconds being achieved. The computer systems required for this are known to a person skilled in the art. The transformed image volume (the breast which is decompressed virtually) can be displayed as a surface mesh or a three-dimensional volume with a constant intensity (voxel value) within the breast and with another intensity outside of the breast, with the three-dimensional volume with the variable intensity within the breast corresponding to the breast density or the voxel intensity values that were estimated from the DBT or FFDM view(s). The lesions can be displayed as color-coded and/or shape-coded objects of the same size, or with sizes that correspond to the dimensions of the lesions that were measured manually or calculated automatically from one or more of the DBT and/or FFDM view(s).

The views of the breast which is decompressed virtually, i.e. of the transformed image volume, can be stored individually or as a set of files in the DICOM, JPEG, TIFF or any other format. It is possible to display “snapshots” of the surface mesh or the volume reproduction during the rotating display from a set of discrete angles or as a set of slice images, which display the three-dimensional volume with an unchanging intensity (voxel value) within the breast and a different intensity outside of the breast, with the three-dimensional volume with the variable intensity within the breast corresponding to the breast density or the voxel intensity estimated by means of the DBT or the FFDM view(s). It is possible to print out print-outs of the view of the breast which is decompressed virtually, with the location(s) of the lesions being displayed from one viewing angle or from a set of viewing angles.

FIG. 10 shows a medical system 28. The medical system 28 contains an imaging modality 30 with an X-ray beam source 32, a compression plate 34, a compression table 36 and an X-ray detector 38. The X-ray source 32 can be arranged in a pivotable fashion in order to generate projection recordings from different angles, which are captured by the X-ray detector 38.

The projections recorded by the X-ray detector 38 are transmitted to a DBT apparatus 40, where slice recordings are generated that are displayed on the display apparatus 46. A control apparatus 44 can, independently or in conjunction with the DBT apparatus 40, establish changed tissue, which is also displayed on the display apparatus 46. The changed tissue can have cancerous tissue, a carcinoma, a lump or any other medically relevant diagnosis. A radiologist can mark the changed tissue by the input apparatus 48, 50. The transformation apparatus 42 can transform the image data of the compressed breast, generated by the DBT apparatus 40, into decompressed image data, which can for example be displayed on the display apparatus 46 as a mesh representation together with the changed tissue. The control apparatus 44 controls the operation of both the DBT apparatus 40 and that of the transformation apparatus 42.

An advantage of the present invention is that a surgeon can identify the position of the changed tissue in a constellation in which the medically relevant tissue region has a shape that substantially corresponds to the shape of the tissue region during an operation. This can increase the safety of the medical intervention.

When the models are generated or when the models are selected, the thickness of the compressed breast, the size of the breast and/or the tissue density, which is derived from the tissue intensities, are preferably used as selection criteria or as features. The same selection criteria or features are used if a suitable model or breast model should be associated with a patient. Here the tissue density can be determined from the intensities of the tissue in the first image volume.

Finally, reference is made to the fact that the description of the invention and the exemplary embodiments should not, as a matter of principle, be understood as being restrictive in view of a particular physical implementation of the invention. More particularly, a person skilled in the art considers it obvious that the invention can be wholly or partly implemented as software and/or hardware, and/or can be implemented distributed over a plurality of physical products—more particularly also computer program products in this case.

1. A method for visualizing a tissue region, which comprises the steps of:
inserting the tissue region into a capturing region of a first imaging modality, with the tissue region assuming a first shape;
capturing the interior of the tissue region by means of the first imaging modality;
reconstructing a first image volume of the tissue region when the tissue region assumes the first shape; and
performing a first transformation of the first image volume into a second image volume representing the tissue region when the tissue region assumes a second shape.

2. The method according to claim 1, which further comprises:
determining the first image volume assumed by the tissue region in the first shape;
determining one of a tissue density, tissue densities or a tissue density distribution of the tissue region from the first image volume when the tissue region assumes the first shape;
determining a force acting on the tissue region when the tissue region assumes the first shape; and
taking into account the first image volume, the tissue density distribution and the force during the first transformation.

3. The method according to claim 1, which further comprises performing a second transformation of the second image volume into a third image volume, in which the tissue region assumes a third shape.

4. The method according to claim 3, which further comprises:
marking a first partial image volume in the first image volume; and
displaying the first partial image volume at a corresponding position in at least one of the second image volume or the third image volume.

5. The method according to claim 3, wherein the step of the first transformation simulates a decompression of the first image volume or the step of the second transformation simulates a compression of the second image volume.

6. The method according to claim 3, which further comprises generating a mesh representation from at least one of the second image volume or the third image volume.

7. The method according to claim 3, wherein at least one of the step of the first transformation or the second transformation uses a model that is selected from a plurality of models as the model with a best correspondence.

8. The method according to claim 7, wherein the model has at least one of a first image volume model data which is associated with the first shape of the tissue region, a second image volume model data which is associated with the second shape of the tissue region, and a third image volume model data which is associated with the third shape of the tissue region.

9. The method according to claim 7, wherein the model is determined using a plurality of imaging modalities.

10. The method according to claim 7, which further comprises:
extracting characteristic features of the tissue region; and
determining the model on the basis of a change in at least one of a position, an orientation or a size of the characteristic features when the tissue region assumes at least one of the first shape, the second shape or the third shape.

11. The method according to claim 7, which further comprises selecting the model on a basis of characteristic features which includes at least one of a thickness of a compressed breast, a density distribution of the breast or a size of the breast.

12. The method according to claim 1, wherein the tissue region is a mamma.

13. An imaging device for visualizing a tissue region, the imaging device comprising:
a transformation device embodied to:
insert the tissue region into a capturing region of an imaging modality, with the tissue region assuming a first shape;
capture the tissue region by means of the imaging modality;
establish a first image volume of the tissue region when the tissue region assumes the first shape; and
perform a first transformation of the first image volume into a second image volume representing the tissue region when the tissue region assumes a second shape.

14. An imaging system, comprising:
at least one modality; and
a transformation device embodied to:
insert the tissue region into a capturing region of said modality, with the tissue region assuming a first shape;
capture the tissue region by means of said modality;
establish a first image volume of the tissue region when the tissue region assumes the first shape; and
perform a first transformation of the first image volume into a second image volume representing the tissue region when the tissue region assumes a second shape.

15. A computer program product loaded into a memory of a computer, the computer product performing a method for visualizing a tissue region, which comprises the steps of:
inserting the tissue region into a capturing region of an imaging modality, with the tissue region assuming a first shape;
capturing the tissue region by means of the imaging modality;
establishing a first image volume of the tissue region when the tissue region assumes the first shape; and
performing a first transformation of the first image volume into a second image volume representing the tissue region when the tissue region assumes a second shape.
